(19) United States
(12) Patent Application Publication
(10) Pub. No.: US 2022/0105229 A1
(43) Pub. Date: Apr. 7, 2022

(54) CCFL STERILIZING APPARATUS

(71) Applicant: Ray Chen, Chino, CA (US)

(72) Inventor: Ray Chen, Chino, CA (US)

(21) Appl. No.: 17/062,603

(22) Filed: Oct. 4, 2020

(51) Int. Cl. A61L 9/20; B01J 21/06; B01J 35/00

(52) U.S. Cl. CPC A61L 9/205; A61L 2209/12; B01J 35/004; B01J 21/063

(57) ABSTRACT

In one aspect, a sterilizing lamp may include a lamp main body, a photocatalyst coating outside the lamp main body, and a CCFL light tube inside the lamp main body. The photocatalyst can be activated by the CCFL light passing out from the lamp main body. An emission material that generates the CCL rays may be enclosed in an internal space of the CCFL light tube. In one embodiment, the photocatalyst in the present invention is TiO₂-based. For the specific photocatalyst used in the present invention, the most effective CCFL rays to activate the photocatalyst include a first CCFL ray with shorter wavelength and a second CCFL ray with longer wavelength.

### FIELD OF THE INVENTION

The present invention relates to a sterilizing apparatus, and more particularly to a CCFL sterilizing lamp with specific wavelengths to excite the photocatalyst to effectively achieve the goal of sterilization.

### BACKGROUND OF THE INVENTION

Ambient environmental air in a home, office, educational, institutional, industrial or institutional setting can be a contributing factor in maintaining a healthy environment. Particulates, such as pollen, dust, mold, spores, bacteria, viruses, animal dander, skin cells, or the like, and volatile chemicals, including volatile organic compounds, commonly referred to as VOCs, formaldehyde, cleansers, pesticides, fungicides, combustion by-products, odors and toxic gases are frequently present in the ambient air. These airborne elements have been implicated in a wide variety of respiratory conditions and diseases.

Generally, a UV lamp is used in various fields so as to sterilize bacteria and fungus by generating UV rays. As the UV lamp is in the form of a lamp, the UV lamp may be appropriately used with simple manipulation when necessary. Furthermore, installation costs and maintenance costs of the UV lamp are inexpensive, and as UV rays generated by the UV lamp are hardly changed, the UV rays continuously maintain a same sterilizing power.

The UV lamp generates UV rays having various wavelengths according to a material used therein. For example, the UV lamp may generate UV-A (wavelength of 400 nm to 315 nm), UV-B (wavelength of 15 nm to 280 nm), or UV-C (wavelength of 280 nm to 110 nm), for example. Among these wavelength, the UV rays having a wavelength of 2531 nm at a wavelength corresponding to the UV-C have a strongest sterilizing power. When the UV-C is irradiated to a DNA of the bacteria and fungus, the DNA of the bacteria and fungus is damaged and destroyed. That is, the UV rays damage a DNA of a living organism and has an effective sterilizing power with respect to various bacteria.

One approach for treating air involves photocatalytic oxidation (PCO) technology, which has been used to remove organic contaminants and compounds from air fluid streams. In commonly used institutional air filtration systems that incorporate PCO technology, the PCO system used generally include one or more ultraviolet (UV) energy sources for irradiating UV light onto a substrate with a titanium dioxide (TiO₂) coating. Disintegration of organic compounds takes place through reactions with oxygen (O₂) and hydroxyl radicals (OH). The O₂ and OH reactions with VOCs drive these diverse gas-phase odor causing contaminants to change their chemical make-up, thereby reducing odors.

Recently, CCFLs, or “Cold Cathode Fluorescent Lamps,” have been developed, which are a kind of low-pressure mercury discharge lamp. The principle of the CCFLs is the same as that of a common fluorescent lamp, in which a trace of mercury is provided inside an envelope having a layer of phosphors coated therein. By adding a high electric field between electrodes at both ends of the envelope, discharge occurs in the low-pressure mercury vapor. Mercury atoms excited by its discharged electrons emit ultraviolet rays of 253.7 nm, and these ultraviolet-rays excite the phosphors in the envelope. Thus, the CCFLs can be described as a transducer converting electrical energy into light energy. Furthermore, cold means that the electrodes of CCFLs are not heated like in standard neon lamps. The electrodes thereof can be miniaturized and simplified to provide a thin envelope, high illumination, high efficiency, low heat, long life, and stability.

The advantages of CCFLs compared with the hot electrode fluorescent lamps are that they have a very long life (usually) 15000 hours or more) in consequence of their rugged electrodes, lack of filament and low current consumption. They start immediately, even under cold ambient conditions. Their life is unaffected by the number of starts. Also, they may be dimmed to very low levels of light output.

However, when a large-sized UV lamp is installed, a uniform plane may not be uniformly sterilized. Moreover, the effective sterilizing distance for the UV lamp is less than 5 ft, so the large-sized UV lamp has to have high power consumption and the electric charges may be increased due to an increase in power facility expansion costs and power consumption for satisfying power to be consumed. Also, UV rays are believed to damage the DNA of the living organism, so great care is needed not to irradiate the UV rays to people Therefore, there remains a need for a new and improved sterilizing apparatus using CCFLs to overcome the problems presented above.

### SUMMARY OF THE INVENTION

In one aspect, a sterilizing lamp may include a lamp main body, a photocatalyst coating outside the lamp main body, and a CCFL (Cold Cathode Fluorescent Lamp) light tube inside the lamp main body. The photocatalyst can be activated by the CCFL light passing out from the lamp main body. An emission material that generates the CCFL, rays may be enclosed in an internal space of the CCFL light tube. Details of the emission material will be discussed below.

In one embodiment, the lamp main body may be provided to be elongated in a lengthwise direction. A length of the lamp main body may be variously provided according to the usage and user preference. Namely, the lamp main body may have various lengths. In a further embodiment, the lamp main body may be made of a material through which the CCFL rays generated in the internal space may be easily transmitted to the outside to activate the photocatalyst. For example, the lamp main body may be made of quartz, borosilicate, or a glass containing the quartz or the borosilicate, for example. As the quartz has excellent permeability, loss of the CCFL rays may be minimized.

In another embodiment, a sterilizing lamp may include a lamp main body including a lamp cover and a lamp receiving space, and a photocatalyst coating outside the lamp cover. In one embodiment, a spiral CCFL light tube is received inside the circular receiving space. Likewise, the photocatalyst can be activated by the CCFL light passing out from the lamp cover.

More specifically, the lamp main body is circular with the lamp cover coated with photocatalyst, and the spiral CCFL light tube is received inside the circular receiving space that is covered by the lamp cover. It is noted that an emission material that generates the CCFL rays may be enclosed in an internal space, which may be sealed in a state in which the emission material is filled. Therefore, the internal space may form a space where no materials are introduced from the outside.

In one embodiment, the emission material may be provided in a gas state and may further include a small amount of mixture. In another embodiment, the emission material may be a mixture of different emission materials of a gas state. The emission material may include one or more of Hg, Ne, Xe, Kr, Ar, XeBr, XeCl, KrBr, KrCl, etc. Furthermore, except for Hg, all of the emission materials may be present in a gas state; and the materials except for Hg may be referred to as a “charging gas.”

It is noted that among the emission materials Ne, Xe, Kr and Ar may be inert gases which hardly cause a chemical reaction with other elements and may be a material that generates a wavelength in a specific case. Hg may generate UV rays having excellent sterilizing power.

When the emission material is disposed on the electric field, the emission material may be discharged and excited in the closed internal space of the CCFL light tube. When the emission material is discharged and excited, CCFL rays may be generated. A wavelength of the generated CCFL rays may be different according to a type of the emission material enclosed in the lamp main body. In one embodiment, by manipulating the composition of the emission material, a spectrum of the CCFL rays can be obtained, which can activate the photocatalyst to achieve the goal for sterilization.

In a further embodiment, the photocatalyst in the present invention is TiO₂-based. For the specific photocatalyst used in the present invention, the most effective CCFL rays to activate the photocatalyst include a first CCFL ray with shorter wavelength and a second CCFL ray with longer wavelength. More specifically, the wavelength of the first CCFL ray ranges from 382-485 nm including UV and blue light, while the wavelength of the second CCFL ray ranges from 505-550 nm including green and yellow light.

The photocatalyst in the present invention can be first activated by the first CCFL ray with shorter wavelength, and an effective range for this first photocatalyst activation is about 1 to 10 inches from the sterilizing lamp. After being activated by the first CCFL ray with shorter wavelength, the photocatalyst may leave the lamp cover and can be activated again by the second CCFL ray with longer wavelength, which would significantly extend and enhance the sterilizing effect of the sterilizing lamp in the present invention.

### BRIEF DESCRIPTION OF THE DRAWINGS

FIG. 1 is a schematic view of the sterilizing apparatus in the present invention.

FIG. 2 illustrates a schematic view of another embodiment of the sterilizing apparatus in the present invention.

FIG. 3 is a partial exploded view of the embodiment of the sterilizing apparatus in the present invention in FIG. 2.

FIG. 4 is a spectrum of one specific kind of CCFL ray to excite the photocatalyst in the present invention.

FIG. 5 shows experimental results of CCFL lamp in the present invention to effectively delay oxidation process of a banana.

FIG. 6 illustrates experimental results of CCFL lamp in the present invention to effectively remove HCHO in the air.

FIG. 7 shows experimental results of CCFL lamp in the present invention to effectively reduce smokes.

FIGS. 8 and 9 illustrate experimental results of CCFL lamp in the present invention to effectively inhibit contamination.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

As used in the description herein and throughout the claims that follow, the meaning of “a”, “an”, and “the” includes reference to the plural unless the context clearly dictates otherwise. Also, as used in the description herein and throughout the claims that follow, the terms “comprise or comprising”, “include or including”, “have or having”, “contain or containing” and the like are to be understood to be open-ended, i.e., to mean including but not limited to. As used in the description herein and throughout the claims that follow, the meaning of “in” includes “in” and “on” unless the context clearly dictates otherwise.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the embodiments. As used herein, the term “and/or” includes any and all combinations of one or more of the associated listed items.

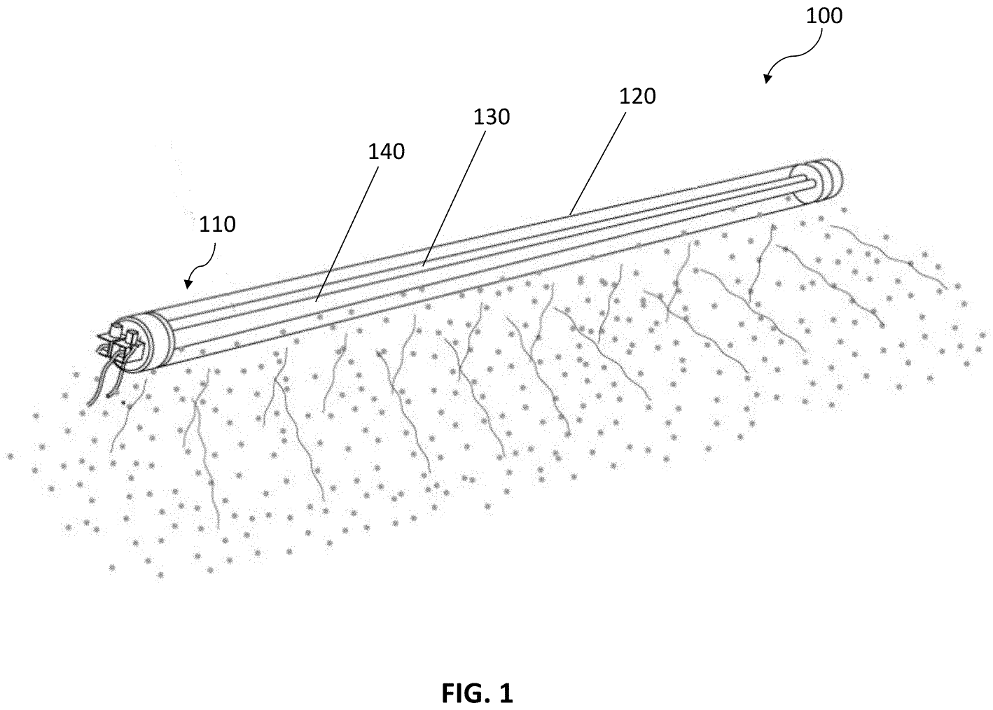

In one aspect, as shown in FIG. 1, a sterilizing lamp 100 may include a lamp main body 110, a photocatalyst coating 120 outside the lamp main body 110, and a CCFL (Cold Cathode Fluorescent Lamp) light tube 130 inside the lamp main body 110. The photocatalyst 120 can be activated by the CCFL light passing out from the lamp main body 110. An emission material 140 that generates the CCFL rays may be enclosed in an internal space of the CCFL light tube 130. Details of the emission material will be discussed below.

In one embodiment, the lamp main body 110 may be provided to be elongated in a lengthwise direction as shown in FIG. 1. A length of the lamp main body 110 may be variously provided according to the usage and user preference. Namely, the lamp main body 110 may have various lengths. In a further embodiment, the lamp main body 110 may be made of a material through which the CCFL rays generated in the internal space may be easily transmitted to the outside to activate the photocatalyst. For example, the lamp main body 110 may be made of quartz, borosilicate, or a glass containing the quartz or the borosilicate, for example. As the quartz has excellent permeability, loss of the CCFL rays may be minimized.

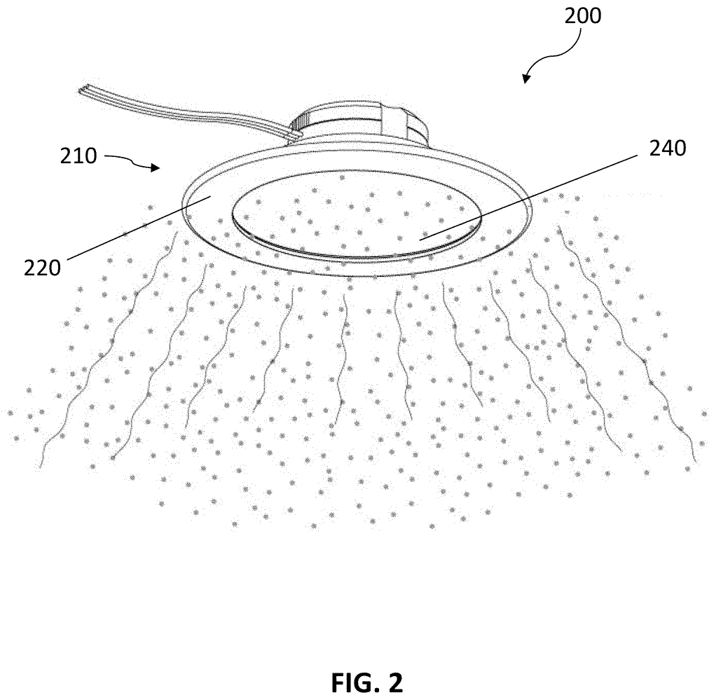

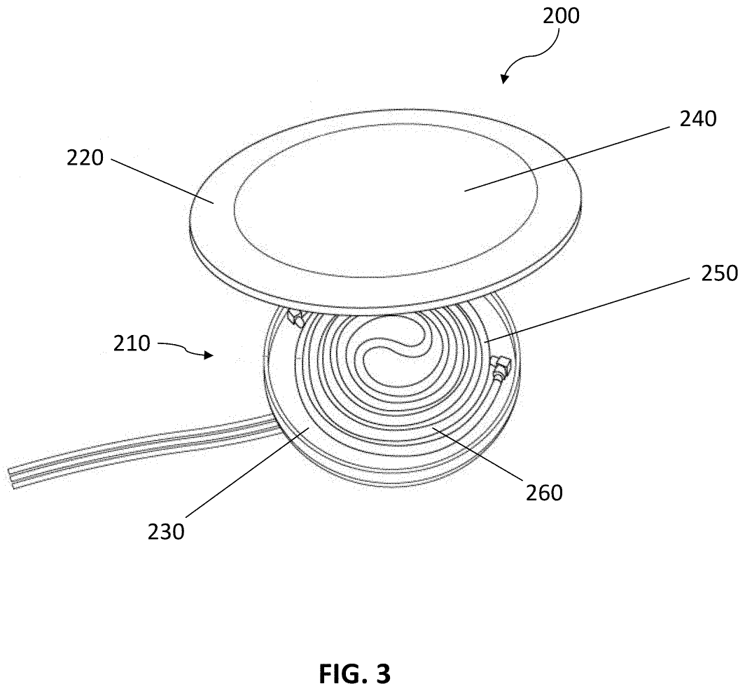

In another embodiment as shown in FIGS. 2 and 3, a sterilizing lamp 200 may include a lamp main body 210 including a lamp cover 220 and a lamp receiving space 230, and a photocatalyst coating 240 outside the lamp cover 220. In one embodiment, a spiral CCFL light tube 250 is received inside the circular receiving space 230. Likewise, the photocatalyst 240 can be activated by the CCFL light 250 passing out from the lamp cover 220.

More specifically, the lamp main body 210 is circular with the lamp cover 220 coated with photocatalyst 240, and the spiral CCFL light tube 250 is received inside the circular receiving space 230 that is covered by the lamp cover 220. It is noted that an emission material (140, 260) that generates the CCFL, may be enclosed in an internal space, which may be sealed in a state in which the emission material (140, 260) is filled. Therefore, the internal space may form a space where no materials are introduced from the outside.

In one embodiment, the emission material (140, 260) may be provided in a gas state and may further include a small amount of mixture. In another embodiment, the emission material (140, 260) may be a mixture of different emission materials of a gas state. The emission material (140, 260) may include one or more of Hg, Ne, Xe, Kr, Ar, XeBr, KrBr, KrCl, etc. Furthermore, except for Hg, all of the emission materials (140, 260) may be present in a gas state, and the materials except for Hg may be referred to as a “charging gas”.

It is noted that among the emission materials (140, 260), Ne, Xe, Kr and Ar may be inert gases which hardly cause a chemical reaction with other elements and may be a material that generates a wavelength in a specific case. Hg may generate UV rays having excellent sterilizing power.

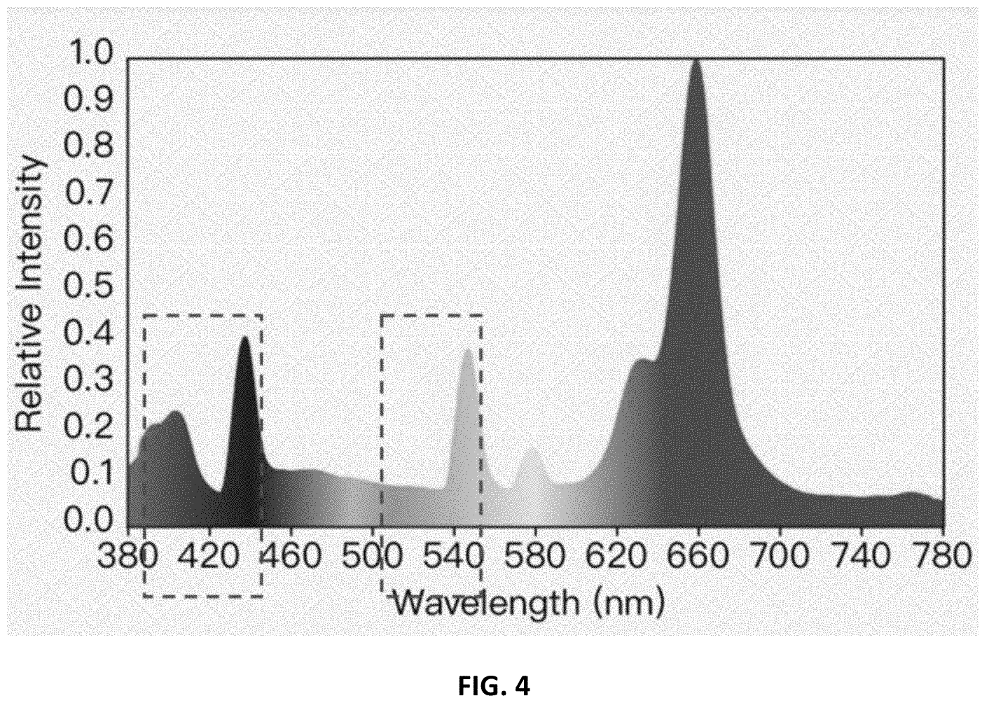

When the emission material (140, 260) is disposed on the electric field, the emission material may be discharged and excited in the closed internal space of the CCFL light tube (130, 250). When the emission material is discharged and excited, CCFL rays may be generated. A wavelength of the generated CCFL rays may be different according to a type of the emission material enclosed in the lamp main body (110, 210). In one embodiment, by manipulating the composition of the emission material, a spectrum of the CCFL rays as shown in FIG. 4 can be obtained, which can activate the photocatalyst (120, 240) to achieve the goal for sterilization.

In a further embodiment, the photocatalyst (120, 240) in the present invention is TiO₂-based. For the specific photocatalyst used in the present invention, the most effective CCFL rays to activate the photocatalyst (120, 240) include a first CCFL ray with shorter wavelength and a second CCFL ray with longer wavelength as shown in FIG. 4. More specifically, the wavelength of the first CCFL ray ranges from 382-485 nm including UV and blue light, while the wavelength of the second CCFL ray ranges from 505-550 nm including green and yellow light. In still a further embodiment, the ratio of UV:blue light:green light:yellow light of the effective CCFL rays in the present invention can be: 1:9:15:2.

The photocatalyst (120, 240) in the present invention can be first activated by the first CCFL ray with shorter wavelength, and an effective range for this photocatalyst activation is about 1 to 10 inches from the sterilizing lamp (100, 200). After being activated by the first CCFL ray with shorter wavelength, the photocatalyst may leave the lamp cover (120, 220) and can remain activated due to the existence of the second CCFL ray with longer wavelength, which would significantly extend and enhance the sterilizing effect of the sterilizing lamp in the present invention. In short, the first CCFL ray with shorter wavelength can initiate the activation of the photocatalysts (120, 240), while the second CCFL with longer wavelength can keep the photocatalysts (120, 240) in the activated status to extend and enhance the sterilizing effect.

### Experiments

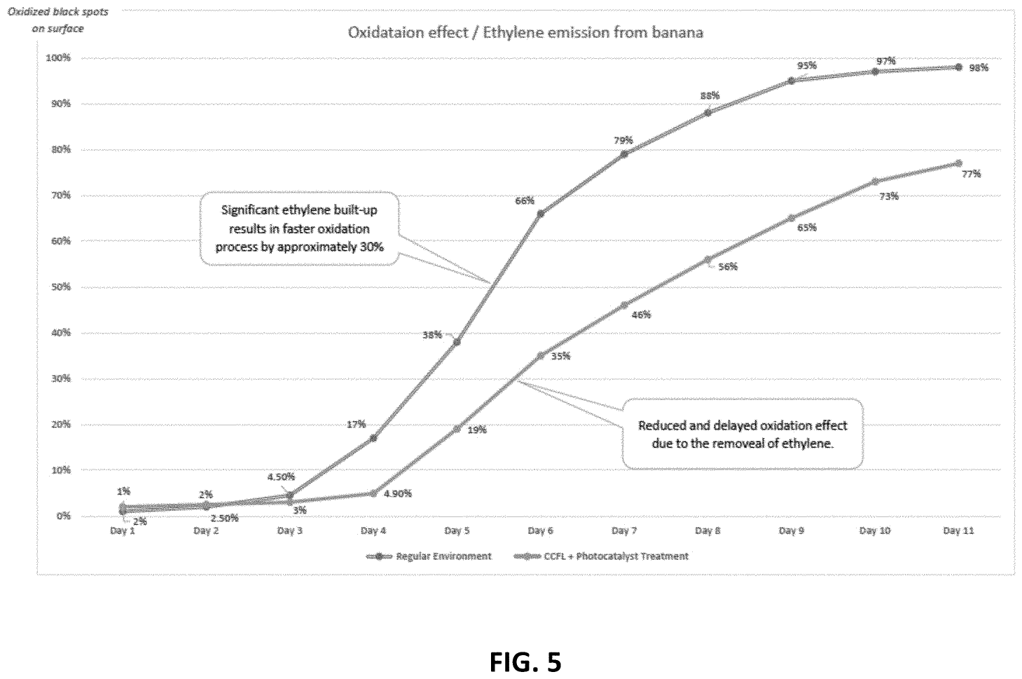

It is believed that ethylene causes yellow pigments in a banana to decay and increases the oxidation process of the banana. The experiment was conducted for eleven days to determine whether the CCFL lamp along with photocatalyst in the present invention can reduce the ethylene level in the air to delay the oxidation process of the banana. FIG. 5 shows the oxidation effect of two bananas: one is treated with the CCFL lamp with photocatalyst coating in the present invention, and the other one is treated with the CCFL lamp without any photocatalyst coating. Since the photocatalyst can be effectively excited by the CCFL lamp in the present invention to reduce the ethylene level in the air, the oxidation process of the banana under the CCFL lamp with photocatalyst coating is much slower than the banana under the CCFL lamp without any photocatalyst coating.

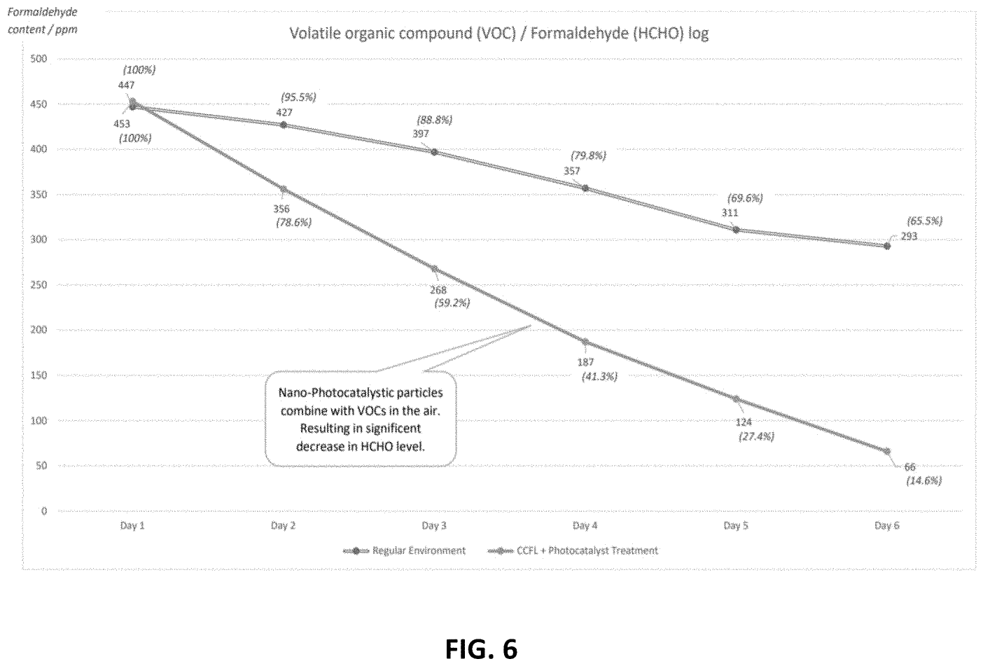

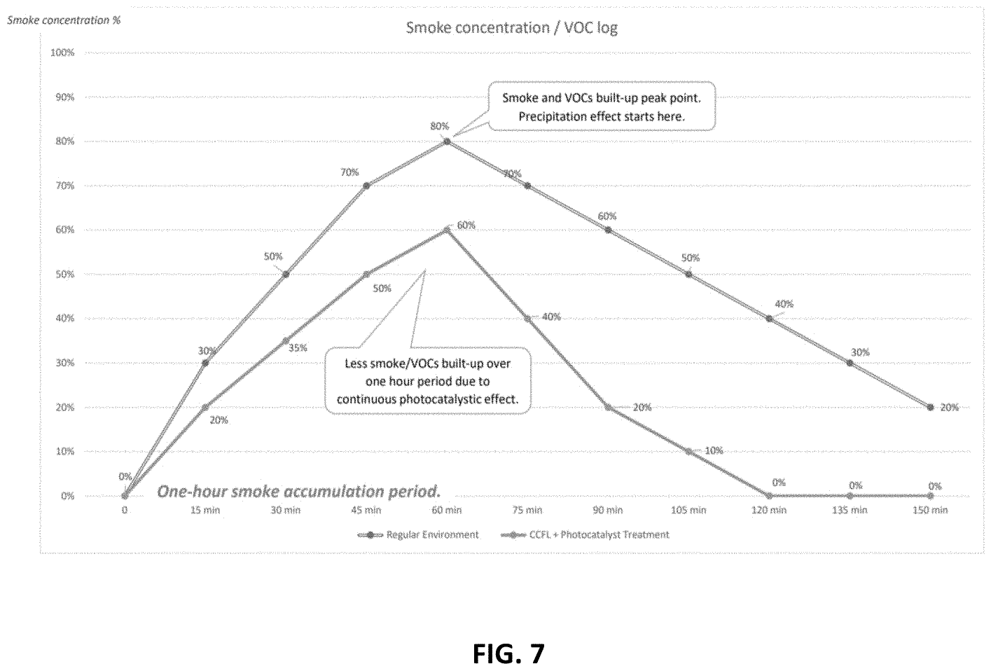

In addition to ethylene, the CCFL rays in the present invention can effectively remove volatile organic compounds (VOCs) such as formaldehyde (HCHO). The experiment was conducted for six days to measure the HCHO level under the CCFL lamp without any photocatalyst coating and the CCFL lamp with the photocatalyst coating in the present invention. As shown in FIG. 6, the HCHO level only reduces 35% under the CCFL lamp without photocatalyst in six days, while the HCHO level reduces about 85% under the CCFL lamp in six days. The results again show the CCFL lamp with specific short and long wavelengths to excite the photocatalyst in the present invention can effectively remove VOCs in the environment. The CCFL lamp along with the photocatalyst in the present invention can also be used to reduce smoke. As shown in FIG. 7, the experiment was conducted for 150 minutes and the smoke concentration under the CCFL lamp with the photocatalyst is always lower than that under the CCFL lamp without any photocatalyst coating, and the smoke concentration can go down to zero under the CCFL lamp with the photocatalyst after 120 minutes.

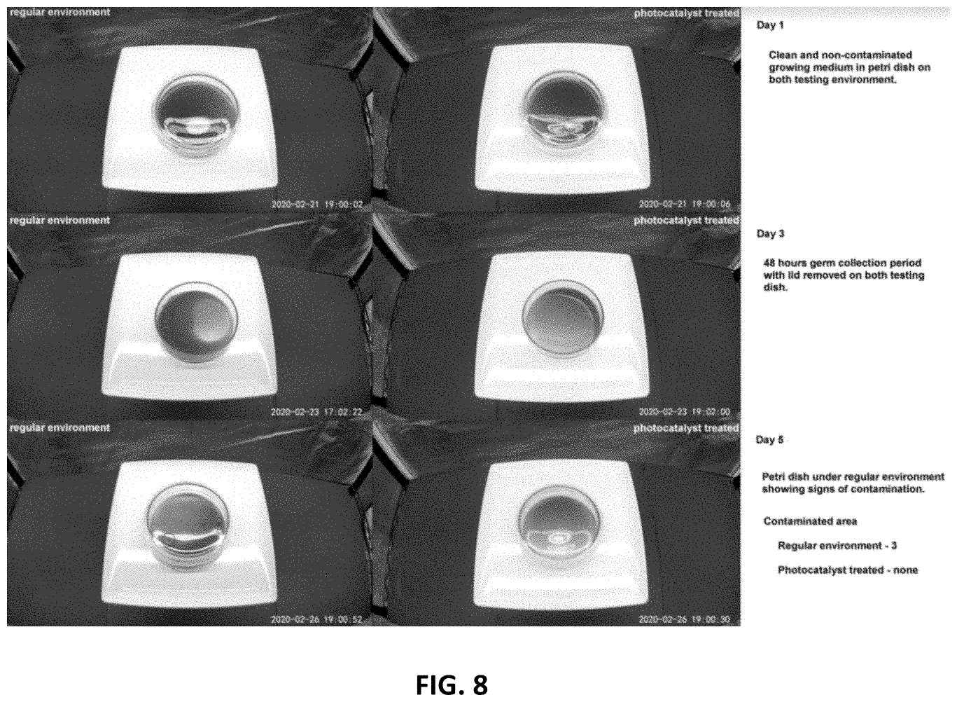

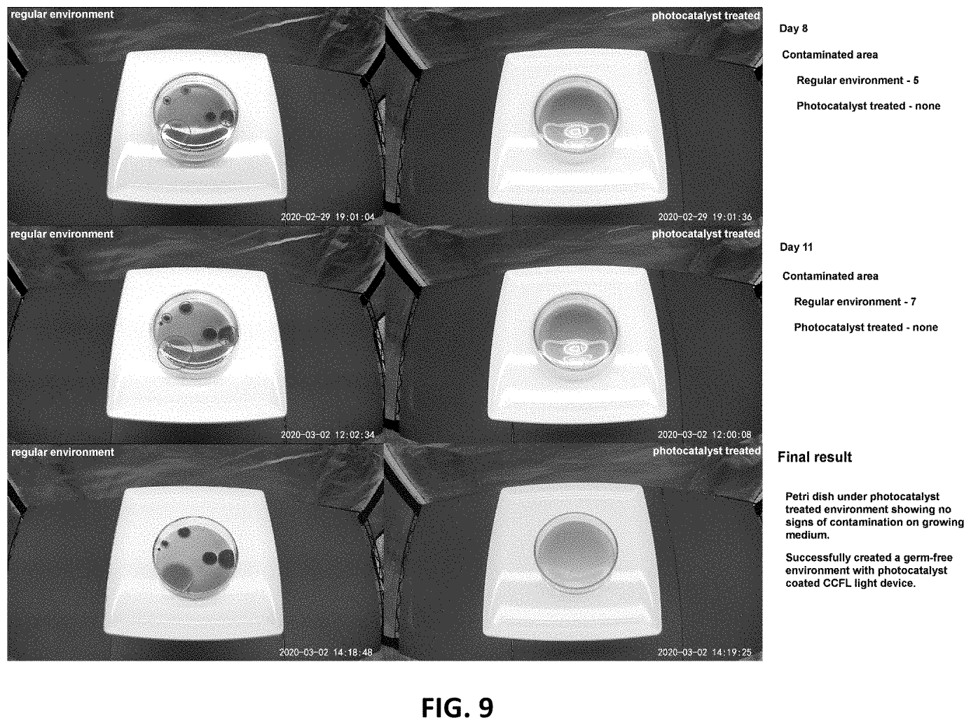

FIGS. 8 and 9 show an eleven-day experiment to treat two petri dishes having identical medium therein. Likewise, one is treated with the CCFL lamp with the photocatalyst in the present invention, and the other one is treated with the CCFL lamp without any photocatalyst. As shown in FIG. 8, for the first 48 hours, there is almost no difference between the two petri dishes. However, starting from the fifth day of the experiment, three black spots indicating contamination in the medium under the CCFL lamp without any photocatalyst were observed, while the medium under the CCFL lamp with photocatalyst in the present invention remained clean. The medium under CCFL lamp with photocatalyst in the present invention still remained clean until the end of the experiment (the 11^{th} day), while the area of the black spots increased in the petri dish under the CCFL lamp without any photocatalyst.

Having described the invention by the description and illustrations above, it should be understood that these are exemplary of the invention and are not to be considered as limiting. Accordingly, the invention is not to be considered as limited by the foregoing description, but includes any equivalent.

What is claimed is:

1. A sterilizing lamp comprising:
a lamp main body;
a photocatalyst coating outside the lamp main body;
a CCFL (Cold Cathode Fluorescent Lamp) light tube inside the lamp main body to generate CCFL rays; and
emission materials that can be excited, by said CCFL rays enclosed in an internal space of the CCFL light tube;
wherein the emission materials are excited to generate effective CCFL rays including two or more different CCFL rays with predetermined wavelengths to active the photocatalyst in at least two predetermined distances to enhance the sterilizing effect.

2. The sterilizing lamp of claim 1, wherein the wavelengths of the CCFL rays to effectively activate the photocatalyst are 382-485 nm and 505-550 nm.

3. The sterilizing lamp of claim 2, wherein the photocatalyst can be first activated by the 382-485 nm CCFL rays between 1 to 10 inches from the sterilizing lamp, and then activated by the 505-550 nm CCFL rays beyond 10 inches from the sterilizing lamp.

4. The sterilizing lamp of claim 1, wherein the emission materials include one or more of Hg, Ne, Xe, Kr, Ar, XeBr, XeCl, KrBr and KCl.

5. The sterilizing lamp of claim 1, wherein the photocatalyst is TiO₂-based.

6. The sterilizing lamp of claim 3, wherein the photocatalyst is TiO₂-based.

7. The sterilizing lamp of claim 1, wherein the lamp main body is made of quartz, borosilicate, or a glass containing the quartz or the borosilicate.

8. The sterilizing lamp of claim 1, wherein the lamp main body is elongated.

9. The sterilizing lamp of claim 1, wherein the lamp main body is spiral and received in a lamp receiving space.

10. The sterilizing lamp of claim 1, wherein a ratio of UV light:blue light:green light:yellow light of the effective CCFL rays in the present invention can be: 1:9:15:2.
